(19) United States
(12) Patent Application Publication
(10) Pub. No.: US 2013/0041385 A1
(43) Pub. Date: Feb. 14, 2013

(54) APPARATUS FOR SKIN REDUCTION

(71) Applicant: Joseph Giovannoli, Park Ridge, NJ (US)

(72) Inventor: Joseph Giovannoli, Park Ridge, NJ (US)

(21) Appl. No.: 13/569,783

(22) Filed: Aug. 8, 2012

Related U.S. Application Data
(60) Provisional application No. 61/521,879, filed on Aug. 10, 2011.

(51) Int. Cl. A61B 17/3209; A61B 17/322

(52) U.S. Cl. 606/131; 606/167

(57) ABSTRACT

An apparatus for making an incision in skin and other body tissue includes an incising assembly having a resilient blade assembly and a fingerwheel for rotatably driving the blade assembly, and a spreader disposed within the blade assembly for deforming the resilient blade assembly.

### RELATED APPLICATIONS

This application claims the benefit of U.S. Provisional Application No. 61/521,879, filed Aug. 10, 2011, the entire disclosure of which is incorporated herein by reference.

### FIELD

The present disclosure relates to surgical instruments for skin and tissue reduction or tightening. More particularly, the present disclosure relates to an apparatus for making an incision in skin to eliminate skin wrinkles and laxities with minimal scarring and deformation after healing.

### BACKGROUND

Conventional face-lifts and other types of rhytidectomies, which tighten skin on the face, arms and other parts of the body, rely on relatively large-scale skin removal to reduce skin-area. The surgical site for skin removal is selected so that incision scaring is as imperceptible as possible, and in face-lifts, the surgical site is created away from the location of the unattractive excess skin.

In a conventional face-lift, the surgeon makes an incision with a scalpel proximate the area of skin removal to create an isolated piece of skin that is lifted and removed with the scalpel or a scissors. The remaining, unattractive excess skin is then stretched toward the area where the skin has been removed. An unfortunate drawback of placing the incision remote from the specific area of skin laxity is that more skin than is necessary is stretched to reduce skin laxity or wrinkles. Such stretching reduces skin thickness and limits the number of times a conventional face-lift can be performed. Further, large scale pulling of the skin toward the hairline can, in some cases, give the face an artificial look considered by many to be characteristic of apparent or repeated face-lifts. Still further, conventional face-lifts do not always resolve wrinkling in the mouth and chin areas and conventional rhytidectomies do not lend themselves to resolving problems in other areas of the body including, but not limited to, the junction of thumb and forefinger.

Improved methods for performing face-lifts and other types of rhytidectomies are disclosed in U.S. patent application Ser. Nos. 11/175,004, 11/485,752, and 11/490,663, which disclose methods and apparatus for skin area reduction and tightening using a plurality of small incisions made at or near the location of the unattractive excess skin. The small incisions may be made in a substantially elliptical or navicular shape, and are sufficiently reduced in size so that, when healed, their corresponding scars are virtually imperceptible. The substantially elliptical shape eliminates the “dog ears” that form at the ends of a closed circular incision. In addition, the small, plural incisions remove a sufficient amount of skin to achieve a desired skin-area reduction. The apparatus disclosed in the aforementioned US. patent applications may comprise an array of punches arranged to collectively form an operational region that matches a patch of skin to be treated. Each of the punches may have an elliptical or navicular shape and may comprise a hollow body inside which a rod is slidably accommodated and provided with an adhesive or other gripping arrangement at an end surface for gripping a skin plug incised by the cutting edge.

A holder comprising a housing having a resilient top may be provided for driving the punches when a force is applied to the top. Alternately, the incisions can be made sequentially instead of contemporaneously.

Therefore, an improved apparatus, which is manufacturable and practical, is needed for performing the methods described, for example, in U.S. patent application Ser. Nos. 11/175,004, 11/485,752, and 11/490,663, which reduces skin proximate the wrinkle, laxity or reduction area to be treated, with substantially imperceptible scarring.

### SUMMARY

Disclosed herein is an apparatus for making an incision in skin and other body tissue. In some embodiments the apparatus comprises an incising assembly comprising a blade; and a spreader disposed within the incising assembly for deforming the blade.

In some embodiments the apparatus further comprises a rigid member for maintaining and holding an axis of the spreader in a predetermined position or orientation.

In some embodiments the member comprises a grip.

In some embodiments the spreader is coupled to the member.

In some embodiments the apparatus further comprises a shaft coupled to the member, wherein the spreader is disposed adjacent to or at an end of the shaft.

In some embodiments the spreader comprises one or more bead-like contact members for engaging the incising assembly.

In some embodiments the spreader further comprises one or more spring-like arms that couple the contact members to the shaft.

In some embodiments the spreader comprises a collar, rim, or flange-like member.

In some embodiments the spreader comprises one or more outwardly projecting members.

In some embodiments the projecting members have a cam-like or bead-like shape.

In some embodiments the incising assembly comprises resilient cylindrical wall, wherein the blade is disposed over at least a portion of the wall.

In some embodiments, the apparatus further comprises a fingerwheel for rotating the blade.

In some embodiments the fingerwheel assembly is rotatably coupled to the member.

In some embodiments the incising assembly comprises a projection or textured gripping arrangement for extracting skin or other body tissue.

In some embodiments the apparatus further comprises an ejector extending at least partially through the incising assembly for ejecting skin or other body tissue from the incising assembly.

In some embodiments the blade is resilient and the spreader deforms the resilient blade into a substantially elliptical, oval, or navicular shape, or any other shape that can be formed by applying a force to the blade.

In some embodiments the spreader expands the blade in a first direction while contracting the blade in a second direction orthogonal to the first direction.

In some embodiments the apparatus further comprises a removable cap disposed over the blade, the cap for preventing the spreader from deforming the blade until the cap is removed therefrom.

In some embodiments, the apparatus comprises an incising assembly comprising a resilient blade assembly and a fingerwheel for rotatably driving the blade assembly; and a spreader disposed within the blade assembly for deforming the blade assembly.

In some embodiments the incising assembly can be axially moved.

In some embodiments, the apparatus further comprise a driver for driving the incising assembly.

In some embodiments, one of the blade and spreader is rotatably moveable relative to the other one of the blade and spreader.

### BRIEF DESCRIPTION OF THE DRAWINGS

FIG. 1 is a sectional side view of an embodiment of an incising apparatus of the present disclosure.

FIG. 2A is sectional side view of an embodiment of a handle of the incising apparatus of the present disclosure.

FIG. 2B is a side elevational view of an embodiment of a spreader assembly of the incising apparatus of the present disclosure.

FIG. 2C is a sectional side elevational view of an embodiment of an incising assembly of the incising apparatus of the present disclosure.

FIG. 2D is an enlarged sectional side view of an embodiment of a blade assembly section of the incising assembly.

FIG. 2E is an enlarged sectional side view of the blade assembly section of the incising assembly after removal of a protective cap.

FIG. 3 is a bottom view of a tubular blade of the blade assembly section before and after being deformed by a spreader of the spreader assembly.

FIGS. 4A and 4B are sectional side views of another embodiment of the incising apparatus of the present disclosure where FIG. 4A shows a skin plug ejector of the apparatus in an incising or inactive position and FIG. 4B shows the skin plug ejector in an ejection position.

FIG. 5A is sectional side view of an embodiment of the handle of the incising apparatus shown in FIGS. 4A and 4B.

FIG. 5B is a sectional side elevational view of an embodiment of the incising assembly of the incising apparatus of FIGS. 4A and 4B.

FIG. 6A is a bottom view of an embodiment of the spreader of a spreader/ejector assembly of the apparatus shown in FIGS. 4A and 4B.

FIG. 6B is a side view of the spreader shown in FIG. 6A.

FIG. 7 is a sectional side view of a further embodiment of the incising apparatus of the present disclosure.

### DETAILED DESCRIPTION

The apparatus of the present disclosure, in some embodiments, may combine a very thin-walled tubular incising member or resilient blade with a supportive, resilient, and elastic material, to provide both a sharp cutting edge and the resilient flexibility needed to deform into a substantially elliptical, oval, or navicular shape, or any other shape that can be formed by applying a force to the resilient blade with a relatively low spreading force. The apparatus is intended, without limitation, to be used for performing the skin reducing and tightening methods described in U.S. patent application Ser. Nos. 11/175,004, 11/485,752, and 11/490,663, the disclosures of which are incorporated herein by reference.

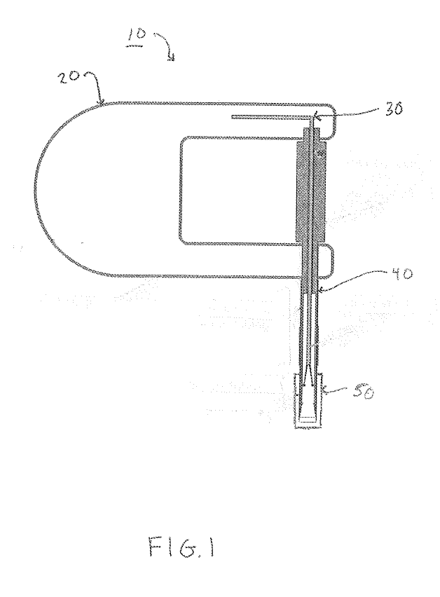

FIG. 1 is a sectional side view of an embodiment of the incising apparatus of the present disclosure, denoted by reference numeral 10. The apparatus 10 may comprise a handle 20, a spreader assembly 30, an incising assembly 40, and a protective cap 50.

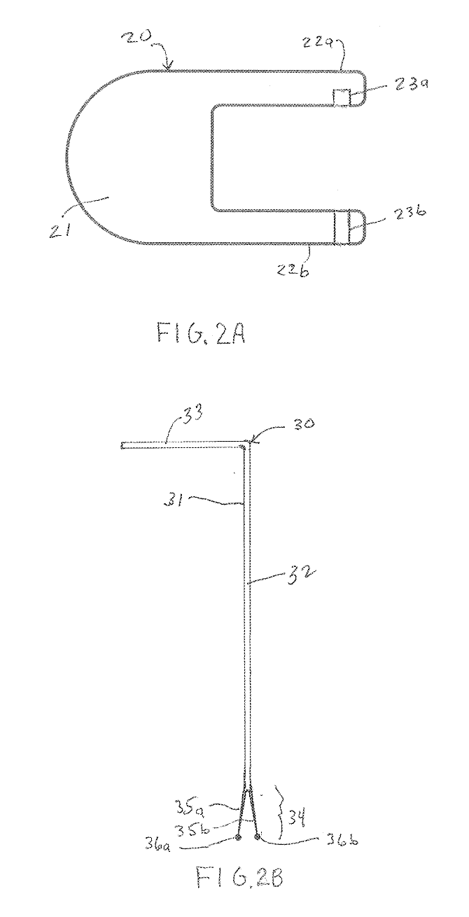

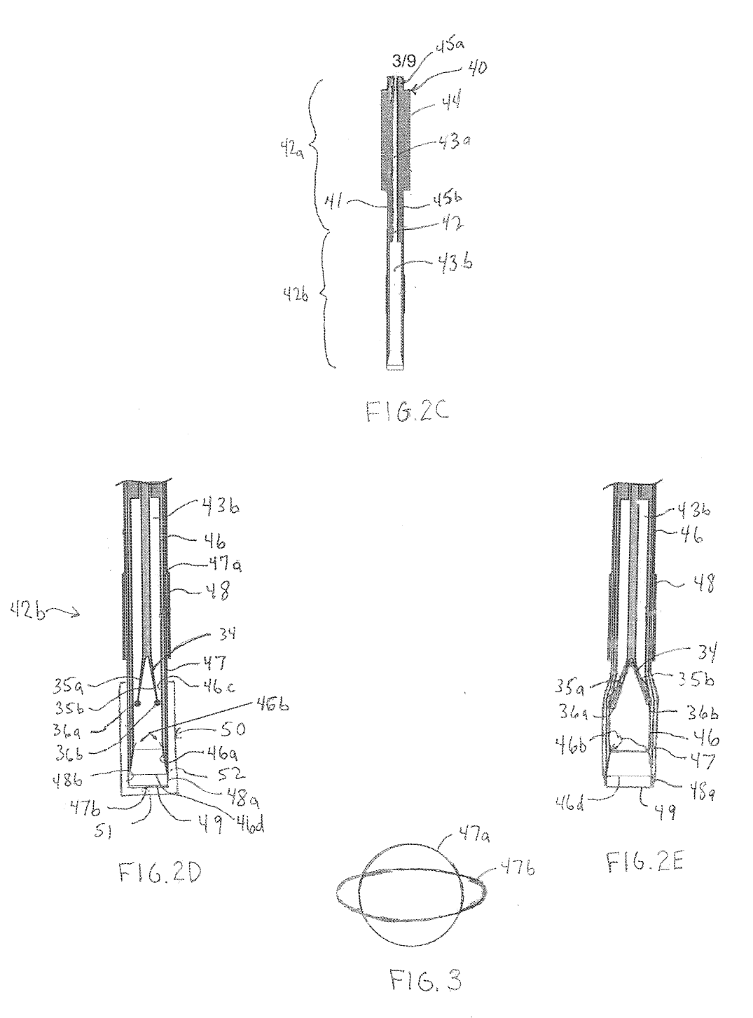

As shown in FIG. 2A, the handle 20 may comprise a grip 21 for gripping and holding the apparatus 10, and spaced apart first and second parallel arms 22a and 22b extending from the grip 21. The grip 21 may have a clam-shell shape, which allows the apparatus 10 to be securely and easily held during the operation of the apparatus 10. In one or more other embodiments, the grip 21 may have any other suitable shape that facilitates gripping and handling of the apparatus 10 during use and/or maintains and holds the axis of the spreader in a predetermined position or orientation.

Referring still to FIG. 2A, the first and second arms 22a and 22b of the handle 20 may include axially aligned first and second apertures 23a and 23b, respectively, which face one another. The first aperture 23a may extend partially or entirely through the first arm 22a while the second aperture 23b extends entirely through the second arm 22b. The apertures 23a, 23b function like bearings for the incising assembly 40, as will be explained further on.

Referring to FIG. 2B, the spreader assembly 30 may comprise an L-shape cylindrical rod or shaft 31 defined by an elongated body 32 and a foot 33 extending orthogonally to the body 32 at a first end thereof. A spreader 34 may be formed at a second end of the elongated body 32. The spreader 34 may include first and second spring-like arms 35a and 35b that depend from the second end of the elongated body 32. Bead-like contact members 36a and 36b may be disposed at the free ends of the first and second spring-like arms 35a and 35b, respectively. The bead-like contact members 36a, 36b define sliding surfaces for engaging the incising assembly 40, as will be described further on.

The foot 33 of the spreader assembly 30 may be fixedly disposed within the first arm 22a of the handle 20. The elongated body 32 of the spreader assembly 30 may extend through the centers of the first and second apertures 23a and 23b of the first and second arms 22a and 22b, respectively.

Referring to FIG. 2C, the incising assembly 40 may comprise a rotatable shaft 41 made of a resilient, elastically deformable material having a fingerwheel section 42a and a blade assembly section 42b. The rotatable shaft 41 can be made of nylon or any other suitable resilient, elastically deformable material. The shaft 41 may include a longitudinally stepped axial bore 42 having small and large diameter portions 43a and 43b, respectively. The small diameter portion 43a of the stepped bore 42 may extend through the fingerwheel section 42a and allows the elongated body 32 of the spreader assembly 30 to pass therethrough into the large diameter portion 43b. The large diameter portion 43b of the stepped bore 42 may extend through the blade assembly section 42b of the incising assembly 40 to accommodate the spreader 34 of the spreader assembly 30.

The fingerwheel section 42a includes a fingerwheel 44. The fingerwheel 44 may include a first cylindrical journal 45a extending from a first end thereof, and an elongated, second cylindrical journal 45b extending from a second end thereof, which merges with the blade assembly section 42b. The outer surface of the fingerwheel 44 may be knurled to allow the fingerwheel 44 to be gripped and rotated between two fingers. The fingerwheel 44 may be rotatably disposed between the first and second arms 22a and 22b of the handle 20 with the first cylindrical journal 45a rotatably disposed within the first aperture 23a of the first arm 22a and the elongated, second cylindrical journal 45b rotatably disposed within the second aperture 23b of the second arm 22b and extending therethrough.

FIG. 2D is an enlarged sectional view of the blade assembly section 42b of the incising assembly 40. The blade assembly section 42b and the large diameter portion 43b of the stepped bore 42 define a thin cylindrical wall 46, which is resilient, and elastically deformable. The cylindrical wall 46 may have a circular cross-sectional profile in an un-deformed state. The cylindrical wall 46 may depend from the second cylindrical journal 45b of the fingerwheel 44. The free end of the cylindrical wall 46 may have a beveled inner edge surface 46a. The innermost edge of the beveled surface may define a skin extracting member or members 46b. The skin extracting member(s) 46b operates to extract a plug of skin generated by the incising assembly 40, and to prevent the plug of skin from slipping under the dermal layer. In one or more embodiments, the skin extracting member 46b may comprise a proximally extending circular gripping ridge. In one or more other embodiments, the skin extracting member 46b may comprise one or more proximally extending discrete burrs or projections. In one or more further embodiments, the skin extracting member 46b may comprise a textured gripping arrangement defined or formed on the inner surface 46c of the cylindrical wall 46.

Referring still to FIG. 2D, the blade assembly section 42b may further comprise a thin, flexible, and tubular surgical blade 47 having first and second opposing ends 47a and 47b, respectively. The second end 47b of the blade 47 may have a beveled outer edge surface 48a that defines a cutting edge 49 with an inner surface 48b of the blade 47. In other embodiments, the second end 47b of the blade 47 may have a beveled inner edge surface (not shown) or beveled inner and outer edge surfaces. A substantial portion of the tubular blade 47 may overlap and surround a portion of the cylindrical wall 46, thereby forming a composite wall. The overlapping tubular blade 47 and cylindrical wall 46 portions may be disposed laterally about the spreader 34. The beveled outer edge surface 48a and the cutting edge 49 of the blade 47, may extend beyond a free edge 46d of the cylindrical wall 46. Because the tubular blade 47 is thin and flexible, it conforms to the shape of the cylindrical wall 46 before and after it is elastically deformed by the spreader 34. A locking collar 48 may be provided for securing the tubular blade 47 to the cylindrical wall 46. The locking collar 48 may surround and engage the upper portion of the tubular blade 47 and an upper portion of the cylindrical wall 46, to prevent separation of the tubular blade 47 from the cylindrical wall 46. In one or more embodiments, the locking collar 48 may comprise a section of heat shrinkable Teflon tubing. In one or more other embodiments, the locking collar 48 may be replaced by a mechanical coupling (e.g., one or more dimples formed in an inner surface of the tubular blade 47 which engage corresponding depressions formed on the outer surface of the cylindrical wall 46) or an adhesive disposed between the tubular blade 47 and the cylindrical wall 46 in the area above the spreader 34.

Referring still to FIG. 2D, a marginal lower end of the large diameter portion 43b of the stepped bore 42 of the cylindrical wall 46 accommodates the spreader 34. The bead-like contact members 36a, 36b of the spring-like spreader arms 35a, 35b engage the inner surface 46c of the cylindrical wall 46 or the resilient tubular blade 47. The cylindrical wall 46 has a thickness which allows it to support the thin, flexible, and tubular blade 47 during the operation of the apparatus 10 and be elastically deformed by the spring-like arms 35a, 35b of the spreader 34. In one or more embodiments, the thickness of the cylindrical wall 46 is typically about 0.001 to 0.005 inches depending upon the composition and diameter of the blade 47. In one or more other embodiments, the thickness of the cylindrical wall 46 can be less than 0.001 inches or greater than 0.005 inches, depending upon the resilient, elastic properties of the material from which the cylindrical wall 46 is made. The cylindrical wall 46 can be made of nylon or any other suitable supportive, resilient, and elastic material. The thin, flexible, and tubular blade 47 may have a wall thickness of, but not limited to, about 0.5 mils depending upon the composition of the blade, the diameter of the blade, and whether the cylindrical wall is utilized. In one or more preferred embodiments, the tubular blade 47 can be made of a superelastic nickel-titanium alloy called Nitinol. Nitinol is preferred because of its superelastic and shape-memory properties. In one or more other embodiments, the tubular blade 47 can be made of steel or any other suitable surgical material that is capable of flexing. The spreader assembly 30 may be made from spring-steel or any other suitable material with spring-biasing properties, and/or have any other suitable configuration that is capable of spreading and deforming the cylindrical wall 46 and/or tubular blade 47 of the incising assembly 40.

The protective cap 50 is optional, and may comprise a cover wall 51 and a cylindrical side wall 52 having a circular cross-sectional profile extending from the cover wall 51. The side wall 52 of the cap 50 may be rigid relative to the cylindrical wall 46 and blade 47 and have an inner diameter that is sized to allow the cap 50 to be installed over a distal end of the incising assembly 40 to cover the cutting edge 49 of the tubular blade 47. The side wall 52 may have a depth that is sized to allow it to surround the tubular blade 47 and cylindrical wall 46 composite in the area generally lateral to the spreader 34, thereby squeezing the spring-like arms 35a, 35b of the spreader 34 together to maintain the circular cross-sectional profile 47a of the cylindrical wall 46 and tubular blade 47 composite (FIG. 2D).

As collectively shown in FIGS. 2D and 2E, removal of the cap 50 from the distal end of the incising assembly 40 allows the spring-like arms 35a, 35b of the spreader 34 to expand or spread outwardly. As the arms 35a, 35b of the spreader 34 spread apart, the bead-like contact members 36a, 36 slide on the inner surface 46c of the cylindrical wall 46 and elastically deform the lower portion of the tubular blade 47 and cylindrical wall 46 composite (FIG. 2E) thereby transforming the cross-sectional profile of the resilient tubular blade 47 and cylindrical wall 46 composite from circular 47a to substantially elliptical (47b), oval, or navicular shape, or any other shape that can be formed by applying a force to the resilient tubular blade, as shown in FIG. 3. In other embodiments of the apparatus, the spreader may comprise three or more spring-like arms terminated with beadlike contact members. In such embodiments, the spring-like arms of the spreader would expand and elastically deform the tubular blade 47 and cylindrical wall 46 composite into a substantially triangular or square cross-sectional profile, or other cross-sectional profiles, depending upon the number of spring-like arms.

In operation, a surgeon may remove the cap 50 from the blade assembly 42b and place the cutting edge 49 of the tubular blade 47 against the skin to be tightened or treated. The surgeon, may then rotate the fingerwheel 44 with his or her fingers to rotate the blade assembly 42b, thereby generating a substantially elliptical, oval, or navicular shape, or any shaped incision in the skin with the tubular blade 47. When the tubular blade 47 of the incising assembly 40 incises the skin, a skin plug may enter the large diameter portion 43b of the stepped bore 42 of the cylindrical wall 46. The extracting member or members 46b disposed on the inner surface 46c of the cylindrical wall 46 will hold the skin plug inside the large diameter portion 43b of the stepped bore 42 so it can be removed together with the tubular blade 47 when the incising assembly 10 is withdrawn from the skin. This process may be repeated in accordance, for example, with the methods described in U.S. patent application Ser. Nos. 11/175,004, 11/485,752, and 11/490,663.

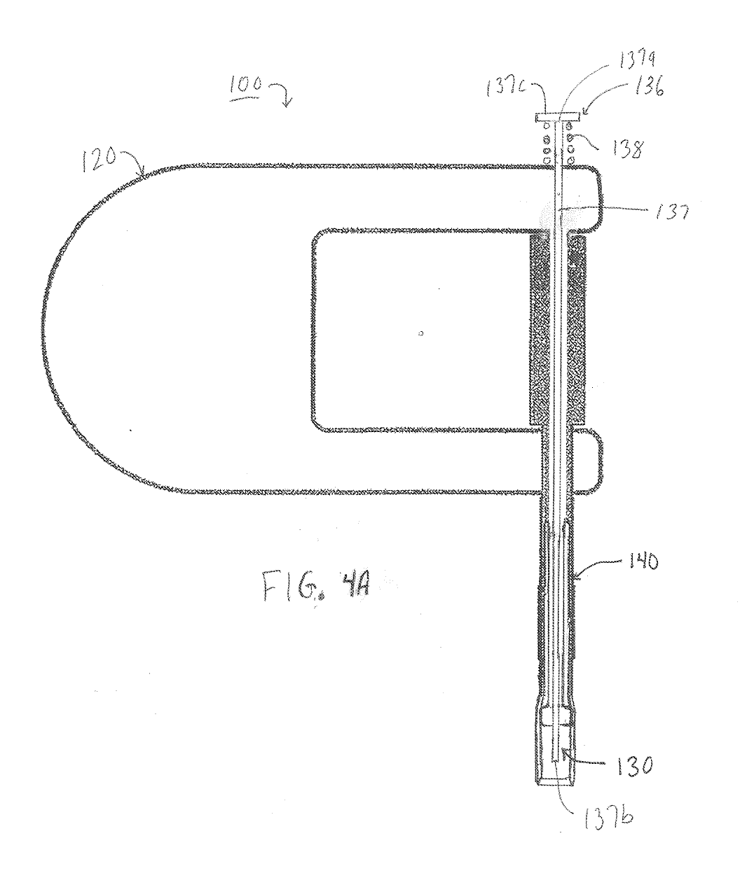

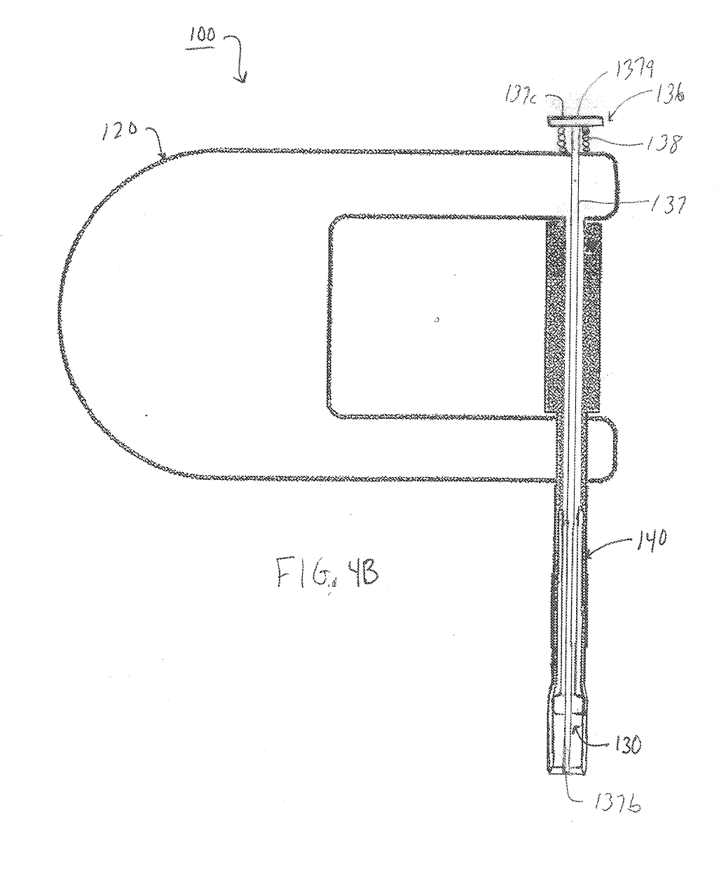

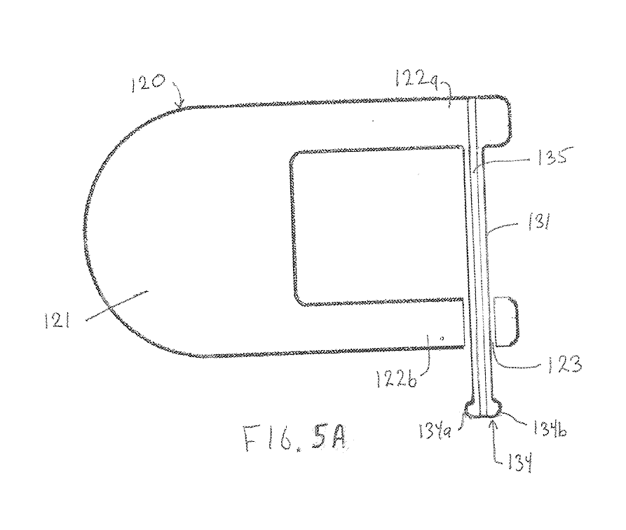

FIGS. 4A and 4B are sectional side views of another embodiment of the incising apparatus of the present disclosure denoted by reference numeral 100. The incising apparatus 100 may comprise a handle 120, a spreader/ejector assembly 130, and an incising assembly 140.

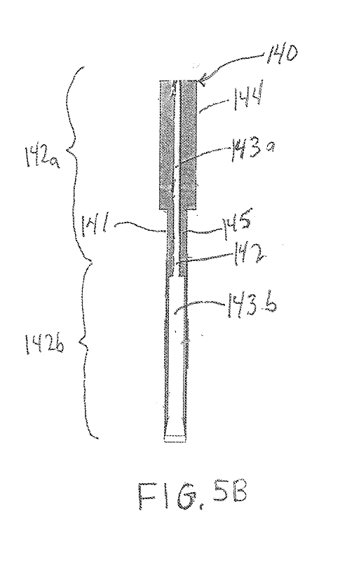

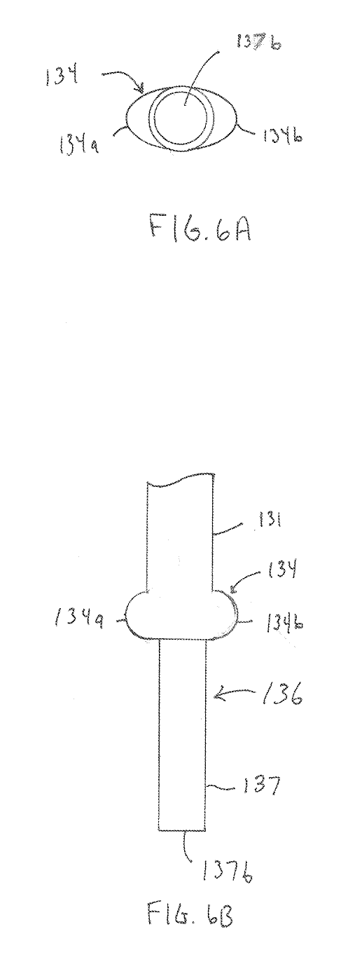

As shown in FIG. 5A, the handle 120 may comprise a clam-shell or other suitably shaped grip 121 for gripping and holding the apparatus 100, spaced apart first and second parallel arms 122a and 122b extending from the grip 121, and an elongated pin or shaft 131 of the spreader/ejector assembly 130. The shaft 131 may depend from the first arm 122a and be unitary with or separate from the first arm 122a. The shaft 131 may extend through an aperture 123 defined in the second arm 122b and be cylindrical in shape so that it can function as an axle for the incising assembly 140. A spreader 134 may be formed at a free end of the shaft 131. The spreader 134 may comprise a collar, rim, or flange-like member having a substantially elliptical, oval, navicular or other desired shape. The spreader 134 may define opposing, outwardly projecting portions 134a and 134b. In other embodiments, the spreader 134 may define more than three outwardly projecting portions. A bore 135 may extend transversely through the first arm 122a of the handle 120, and continue through the shaft 132 and spreader 134.

Referring again to FIGS. 4A and 4B, the spreader/ejector assembly 130 also includes a skin plug ejector 136 for pressing out any skin plugs that are generated by and retained within the blade assembly section 42b. The ejector 136 may comprise an elongated rod 137 having first and second ends 137a and 137b, respectively. A finger pad 137c may be disposed at the first end 137a thereof. A return spring 138 may be disposed between the finger pad 137c and the first arm 122a.

As shown in FIG. 5B, the incising assembly 140 of incising apparatus 100 is substantially identical to the incising assembly 40 of incising apparatus 10 except, the fingerwheel 144 of the incising assembly 140 may include a single cylindrical journal 145 disposed at the second end of the fingerwheel 144 that is rotatably disposed within the aperture 123 of the second arm 22b. In addition, the incising assembly 140 may rotate on the shaft 132 of the spreader/ejector assembly 130, which extends through the small diameter portion 143a of the stepped bore 142, and operates as an axle for the incising assembly 140.

As shown in FIGS. 6A and 6B, the opposing projecting portions 134a, 134b of the spreader 134 define convex outer surfaces for engaging an inner surface 146c of a cylindrical wall 146 of the incising assembly 140 and elastically deforming the lower portion of the tubular blade 147 and cylindrical wall 146 composite so that the cross-sectional profile of the tubular blade 147 and cylindrical wall 146 composite has a substantially elliptical, oval, or navicular in shape, or any other shape that can be formed by applying a force to the resilient tubular blade 147 or cylindrical wall 146.

Referring again to FIG. 4A, the rod 137 of the ejector 136 may extend through the bore 135 in the handle arm 122a, shaft 132 and spreader 134. In an incising position, the ejector return spring 138 is in an uncompressed state and positions the ejector 136 within the bore 135 such that the second end 137b of the ejector rod 137 extends just beyond the spreader 134 within the blade assembly section 142b. As shown in FIG. 4B, when the surgeon presses the finger pad 137c of the ejector 136 down into an ejection position to eject a skin plug from within the blade assembly section 142b after incising, the finger pad 137c compresses the ejector return spring 138, and the ejector 136 moves down through the bore 135 so that the second end 137b of the ejector rod 137 moves further down the blade assembly section 142b closer to the cutting edge 149 of the resilient tubular blade 147, to press any skin plug disposed therein out from the blade assembly section 142b.

In some embodiments, the thin cylindrical wall of the incising assembly can be omitted depending upon the composition, the wall thickness and the diameter of the tubular blade.

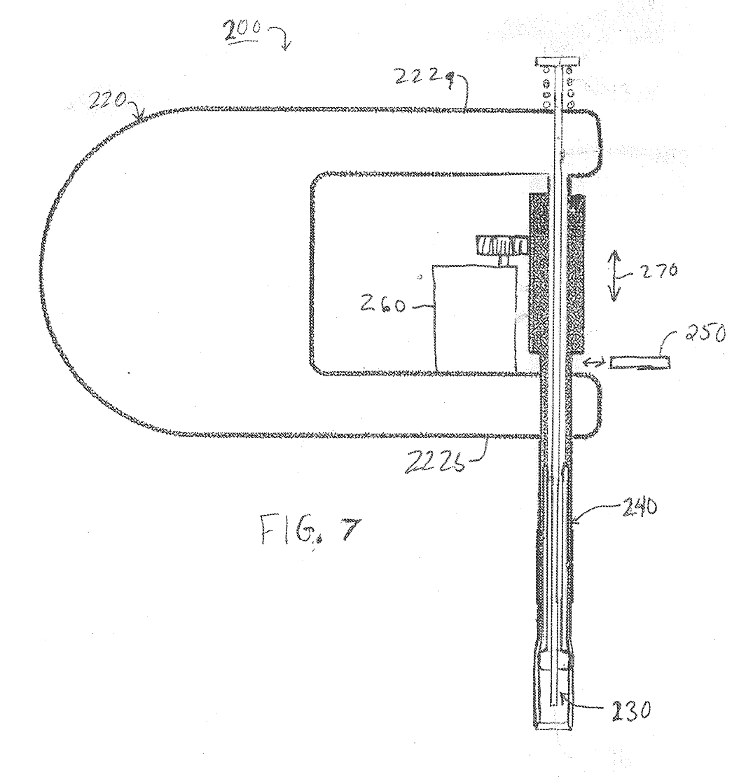

FIG. 7 is a sectional side view of a further embodiment of the incising apparatus of the present disclosure denoted by reference numeral 200. This embodiment is substantially identical to the embodiment shown in FIG. 4A, except that the incising assembly 240 can be axially moved (arrow 270) relative to the spreader 230 to decrease or increase certain dimensions of the resilient tubular blade (e.g., increase the length of the long axis of the substantially elliptical, oval, or navicular shaped blade during deformation of the blade) and/or modify the shape of the tubular blade. Removable U-shaped spacers 250 of different thicknesses may be provided for fixing the axial position of the incising assembly 240 relative to the spreader 230. Specifically, the axial position of the incising assembly 240 may be set or fixed by selecting one of the spacers based on its thickness (which controls the axial position of the incising assembly 240) and inserting it between the fingerwheel 244 and one of the arms 222a, 222b of the handle 220. In other embodiments, a lock screw or other locking arrangement can be used in place of the spacers to prevent axial movement of the incising assembly 240 once an axial position has been selected. An optional driver 260 may be provided for driving the incising assembly 240. The driver 260 may comprise a battery powered motor with a drive wheel that engages and rotates the fingerwheel 244 of the incising assembly 240. In other embodiments, the driver may comprise a wound-spring arrangement or other drive mechanism or means.

While exemplary drawings and specific embodiments of the present disclosure have been described and illustrated, it is to be understood that that the scope of the invention as set forth in the claims is not to be limited to the particular embodiments discussed. Thus, the embodiments shall be regarded as illustrative rather than restrictive, and it should be understood that variations may be made in those embodiments by persons skilled in the art without departing from the scope of the invention as set forth in the claims that follow and their structural and functional equivalents.

What is claimed is:

1. An apparatus for making an incision in skin and other body tissue, the apparatus comprising:
an incising assembly comprising a blade; and
a spreader disposed within the incising assembly for deforming the blade.

2. The apparatus of claim 1, further comprising a rigid member for maintaining and holding an axis of the spreader in a predetermined position or orientation.

3. The apparatus of claim 2, wherein the member comprises a grip.

4. The apparatus of claim 2, wherein the spreader is coupled to the member.

5. The apparatus of claim 2, further comprising a shaft coupled to the member, wherein the spreader is disposed adjacent to or at an end of the shaft.

6. The apparatus of claim 1, wherein the spreader comprises one or more bead-like contact members for contacting the blade or another member of the incising assembly.

7. The apparatus of claim 6, wherein the spreader further comprises one or more spring-like arms that couple the one or more contact members to the shaft.

8. The apparatus of claim 1, wherein the spreader comprises a collar, rim, or flange-like member.

9. The apparatus of claim 1, wherein the spreader comprises one or more outwardly projecting members.

10. The apparatus of claim 9, wherein the projecting members have a cam-like or bead-like shape.

11. The apparatus of claim 1, wherein the incising assembly comprises an elastically deformable cylindrical wall, the blade disposed over at least a portion of the wall.

12. The apparatus of claim 11, further comprising a fingerwheel for rotating the blade.

13. The apparatus of claim 2, further comprising fingerwheel assembly rotatably coupled to the member.

14. The apparatus of claim 1, further comprising a fingerwheel for rotating the blade.

15. The apparatus of claim 1, wherein the incising assembly comprises a projection or textured gripping arrangement for extracting skin or other body tissue.

16. The apparatus of claim 1, further comprising an ejector extending at least partially through the incising assembly for ejecting skin or other body tissue from the incising assembly.

17. The apparatus of claim 1, wherein the spreader deforms the blade into a substantially elliptical, oval, or navicular shape.

18. The apparatus of claim 1, wherein the spreader expands the blade in a first direction while contracting the blade in a second direction orthogonal to the first direction.

19. The apparatus of claim 1, further comprising a removable cap disposed over the blade, the cap for preventing the spreader from deforming the blade until the cap is removed therefrom.

20. The apparatus of claim 1, wherein the blade is resilient.

21. The apparatus of claim 20, wherein the incising assembly includes a wall for supporting the blade.

22. The apparatus of claim 21, wherein the wall is resilient.

23. An apparatus for making an incision in skin and other body tissue, the apparatus comprising:
an incising assembly comprising a resilient blade assembly and a fingerwheel for rotatably driving the blade assembly; and
a spreader disposed within the blade assembly for deforming the blade assembly.

24. The apparatus of claim 23, wherein the spreader deforms the blade assembly into a substantially elliptical, oval, or navicular shape.

25. The apparatus of claim 23, wherein the blade assembly includes a resilient cylindrical wall and a blade disposed over at least a portion of the wall.

26. The apparatus of claim 23, wherein the incising assembly further comprises a projection or textured gripping arrangement for extracting skin or other body tissue.

27. The apparatus of claim 26, wherein the projection or textured gripping arrangement is associated with the blade assembly.

28. The apparatus of claim 23, further comprising an ejector extending at least partially through the incising assembly for ejecting skin or other body tissue from the blade assembly.

29. The apparatus of claim 23, wherein the spreader expands the blade assembly in a first direction while contracting the blade assembly in a second direction orthogonal to the first direction.

30. The apparatus of claim 23, further comprising a removable cap disposed over the blade assembly, the cap for preventing the spreader from deforming the blade assembly until the cap is removed therefrom.

31. The apparatus of claim 23, wherein the spreader comprises one or more bead-like contact members for engaging the blade assembly.

32. The apparatus of claim 31, wherein the spreader further comprises one or more spring-like arms that bias the contact members against the blade assembly.

33. The apparatus of claim 23, wherein the spreader comprises a collar, rim, or flange-like member.

34. The apparatus of claim 23, wherein the spreader comprises one or more outwardly projecting members.

35. The apparatus of claim 34, wherein the one or more projecting members have a cam-like or bead-like shape.

36. The apparatus of claim 1, wherein incising assembly can be axially moved.

37. The apparatus of claim 1, further comprising a driver for driving the incising assembly.

38. The apparatus of claim 1, wherein one of the blade and spreader is rotatably moveable relative to the other one of the blade and spreader.
